(19) United States
(12) Patent Application Publication
(10) Pub. No.: US 2023/0233809 A1
(43) Pub. Date: Jul. 27, 2023

(54) INTRAVENOUS CATHETER

(71) Applicant: Anthony DiCianni, Okemos, MI (US)

(72) Inventor: Anthony DiCianni, Okemos, MI (US)

(21) Appl. No.: 18/130,308

(22) Filed: Apr. 3, 2023

Related U.S. Application Data
(63) Continuation-in-part of application No. 16/916,692, filed on Jun. 30, 2020, now Pat. No. 11,617,863.
(60) Provisional application No. 63/034,312, filed on Jun. 3, 2020.
(60) Provisional application No. 62/947,201, filed on Dec. 12, 2019.
(60) Provisional application No. 62/937,971, filed on Nov. 20, 2019.
(60) Provisional application No. 62/936,516, filed on Nov. 17, 2019.
(60) Provisional application No. 62/923,494, filed on Oct. 19, 2019.

(51) Int. Cl. A61M 25/06; A61M 25/09

(52) U.S. Cl. CPC A61M 25/0606; A61M 25/09; A61M 5/158

(57) ABSTRACT

Intravenous catheter system includes a housing and a guidewire completely disposed within the housing. The system can also include a guide wheel disposed in the housing and operable to rotate about a first axle. The guide wheel can include an inner surface configured to engage the guidewire and move the guidewire. Additionally, the system can include a support wheel operable to engage the guidewire opposite the guide wheel. The support wheel can rotate about a second axle.

### RELATED APPLICATIONS

This application is a continuation-in-part of U.S. application Ser. No. 16/916,692 filed Jun. 30, 2020, which claims priority to and the benefit of U.S. Provisional Patent Application No. 62/923,494 filed Oct. 19, 2019, U.S. Provisional Patent Application No. 62/936,516 filed Nov. 17, 2019, U.S. Provisional Patent Application No. 62/937,971 filed Nov. 20, 2019, U.S. Provisional Patent Application No. 62/947,201 filed Dec. 12, 2019, and U.S. Provisional Patent Application No. 63/034,312, filed Jun. 3, 2020, the entire contents of each of which are hereby incorporated by reference.

### FIELD

The present disclosure relates generally to an intravenous (IV) catheter. In at least one example, the present disclosure relates to an IV catheter that includes a guidewire.

### BACKGROUND

Most IV catheters used today in the United States lack guidewires for IV catheter placement. Some IV catheters do include guidewires.

### BRIEF DESCRIPTION OF THE DRAWINGS

Features of the present disclosure will become apparent to those skilled in the art to which the present disclosure relates from reading the following specification with reference to the accompanying drawings, in which:

FIG. 1 is an elevated perspective view of an intravenous catheter device, according to at least one instance of the present disclosure;

FIG. 2 is an elevated perspective view of the device of FIG. 1 illustrating a typical grip of an operator, according to at least one instance of the present disclosure;

FIG. 3 is a vertical sectional view of the device of FIG. 1, according to at least one instance of the present disclosure;

FIG. 4 is a vertical sectional view of the device of FIG. 1 illustrating insertion into a patient, according to at least one instance of the present disclosure;

FIG. 5 is a vertical sectional view the guidewire of the device disposed further into the patient, according to at least one instance of the present disclosure;

FIG. 6 is a vertical section view along the lines A-A of FIG. 1 with the two wheels and two axles, according to at least one instance of the present disclosure;

FIG. 7 is a vertical sectional view of an alternative version of the device, according to at least one instance of the present disclosure;

FIG. 8 is a vertical sectional view of an alternative version of an example device illustrating insertion into a patient, according to at least one instance of the present disclosure;

FIG. 9 is a view similar to FIG. 8 with the guidewire disposed further into the patient, according to at least one instance of the present disclosure;

FIG. 10 is a vertical sectional view similar to FIG. 6 of the example of FIGS. 7, 8 and 9, according to at least one instance of the present disclosure;

FIG. 11 is a vertical sectional view of a further alternative view, according to at least one instance of the present disclosure;

FIG. 12 is a view similar to FIGS. 3 and 7 of the alternative of FIG. 11 illustrating insertion into a patient, according to at least one instance of the present disclosure;

FIG. 13 is a vertical section view of the alternative of FIG. 11 including an illustration of the patient, according to at least one instance of the present disclosure; and

FIG. 14 illustrates the tensioner and securement element, according to at least one instance of the present disclosure.

### DETAILED DESCRIPTION

It will be appreciated that for simplicity and clarity of illustration, where appropriate, reference numerals have been repeated among the different figures to indicate corresponding or analogous elements. In addition, while specific details are set forth in order to provide an understanding of the examples described herein, it will be appreciated by those skilled in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Several definitions that apply throughout this disclosure will now be presented. The term “coupled” is defined as connected, whether directly or indirectly through intervening components, and is not necessarily limited to physical connections. The term “communicatively coupled” is defined as connected, either directly or indirectly through intervening components, and the connections are not necessarily limited to physical connections, but are connections that accommodate the transfer of data between the so-described components. The connection can be such that the objects are permanently connected or releasably connected. The term “outside” refers to a region that is beyond the outermost confines of a physical object. The terms “comprising,” “including” and “having” are used interchangeably in this disclosure. The terms “comprising,” “including” and “having” mean to include, but not necessarily be limited to the things so described. The term “substantially” is defined to be essentially conforming to the particular dimension, shape or other word that substantially modifies, such that the component need not be exact.

A problem with the older style IV catheters is that it's possible to get a blood flash, indicating that the needle is in the vein lumen, however the plastic catheter that is over the needle is still outside the vein. This is because there is an offset between the distal end of the needle and the start of the plastic catheter that is over the needle. This can lead to inappropriate placement of the catheters and failed IV attempts once the operator tries to advance the plastic catheter when it is partially or completely outside the vein. A potential way to prevent this from happening is using a guidewire that goes through the needle and then advancing the plastic catheter over the guidewire (Seldinger technique).

An issue with the current guidewire assisted intravenous catheters on the market today is a lack of a familiar hand position for advancing the guidewire and catheter with one finger. Many operators who place non-guidewire intravenous catheters do so in a usual fashion of having their thumb and middle finger on each side of the needle grip and their index finger on top of the catheter near a push off tab. Up until the present disclosure, there is no IV catheter on the market with a built-in all-inclusive IV catheter system that allows for an obvious blood flash similar to previous IV catheters, and a front wheel that allows for similar one handed biomechanics for advancing both a guidewire and catheter with the same finger. There is an introducer device on the market that utilizes a front wheel to direct a guidewire, U.S. Pat. No. 10,143,826 B2, however this is an introducer device only that needs to adjoin to another IV catheter and not an all in one IV catheter device. Also, introducer device prevents a blood flash from being visible with many of the IV catheters available on the market, limiting its function to mainly being utilized with ultrasound. Up until now there is no built-in all-in-one IV catheter with a front wheel guidewire system that helps achieve similar biomechanics to previous non-guidewire IV catheters that also allows for a familiar blood flash. The current all-in-one guidewire IV catheter on the market (AccuCath®) has a slide for advancing the guidewire through the needle that cannot be done with one hand alone. The presently available IV catheter also makes it difficult to see blood flash contained within the plastic catheter portion and lacks the more familiar blood flash chamber. With the vast advancement of ultrasound in the last decade, it is important to be able to get an obvious blood flash and then advance an IV guidewire/catheter system with one hand alone so the operator's other hand can simultaneously operate an ultrasound probe. The AccuCath® IV catheter lacks this capability as the ultrasound probe needs to be put down so the operator can advance the guidewire and catheter with their free hand.

Up until now, guidewire assisted intravenous catheters have lacked a blood flash similar to catheters without guidewires. Having a guidewire already in the needle can interfere with blood return ultimately leading to a less obvious flash of blood when the needle enters the vein. The problem with the introducer device, is that the device has to connect directly to the needle blood return system so that the guidewire can advance through the needle, however this prevents a blood flash. This would make using this introducer device impractical if not impossible without an ultrasound machine. The AccuCath® has a difficult to see blood flash within the catheter itself and lacks a traditional blood flash chamber to alert the operator that the needle has entered the vein. An obvious blood flash is important for operators to know that they are in the vein lumen prior to advancing the guidewire, especially without ultrasound guidance.

The solution of the present disclosure to the biomechanics problem of previous guidewire IV catheters is a built-in guide wheel. A guidewire can be located either entirely or partially inside of the housing. The guidewire can be advanced with one finger (for example the index finger, but different fingers are within the scope of this disclosure). Once the guidewire is in the vein, the operator can then use his/her same finger to advance a plastic catheter. In at least one example, a push off tab can be provided on the plastic catheter.

One example of a device according to the present disclosure has a passageway formed in a housing of the device. The device also includes a guidewire that is at least partially contained within the passageway and a guide wheel that extends partially within the housing and is operable to advance the guidewire from the passageway through a needle of the device into a patient. As the guidewire is advanced by rotating the guide wheel, the guidewire can exit the catheter and into a vein of a patient. As the guidewire does not extend past an aperture formed in a needle of the catheter, blood can leave the needle through the aperture into a blood flash chamber, the guidewire does not interfere with blood return into the flash chamber. Therefore, in at least one example, the present disclosure provides for a blood flash that is visible through the housing. Once the blood flash is obtained, the operator can turn the guide wheel, advancing the guidewire further into the needle, past the aperture in the needle, and then into the patient's vein. Once the guidewire is fully inserted, the operator can slide a plastic catheter over the needle and further into a vein lumen. The plastic catheter can remain in the vein of the patient, while the guidewire and needle can be removed from the patient.

The all-inclusive IV catheter of the present disclosure contains a built-in guide wheel for one handed guidewire advancement to help provide proper biomechanics for IV catheter insertion. The present design allows for the complete process to be finished with one hand and the present design allows for a blood flash so that it can be used easily with or without an ultrasound machine. The one-handed technique frees up the operator's other hand. The other hand can be used for interacting with the ultrasound machine therefore allowing the operator to use both the present IV catheter and the ultrasound machine at the same time during the entire procedure.

Once the guidewire has been extended to the proper position. The plastic catheter can be inserted over the guidewire with the same finger that was used to advance the guidewire. The plastic catheter is configured to remain inserted until it is no longer needed. The plastic catheter can have a connection port on the exposed end. The connection port can allow for a future IV connection to be established. The plastic catheter can be removed when the IV port is no longer needed.

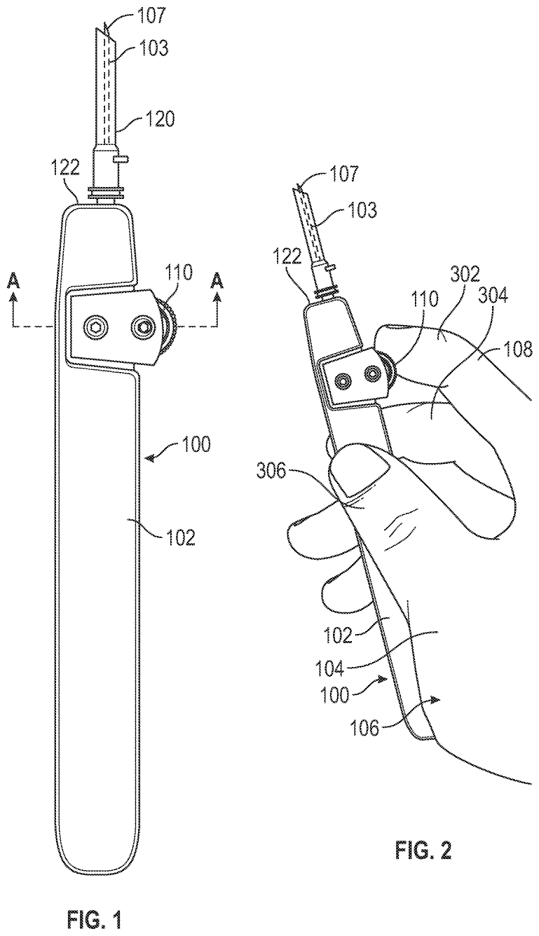

FIG. 1 illustrates an example of the intravenous (IV) catheter guidewire system or device 100 according to the present disclosure. The IV catheter device can be in an outer housing 102. The outer housing 102 can be operable to be handheld. In at least one example, the housing 102, can be made in two pieces. The device 100 can also include a guide wheel 110. As detailed herein, the guide wheel 110 can be used to advance a guidewire. The housing 102 has a distal end 122 that a needle 103 extends from. The needle 103 has a distal end 107 that is located farthest away from the housing 102. The distal end 107 can be tapered or sloped to allow easier penetration of the patient's skin and/or the patient's vein.

The guide wheel 110 can be positioned close to the distal end 122 of housing 102. The distance of the guide wheel 110 from the distal end 122 can be less than 25% of an overall length of the housing 102. In another example, the guide wheel 110 can be located a distance of less than 10% of the overall length of the housing 102 from the distal end 122. The distal end 122 of the housing 102 is the portion of the housing closest to the needle 103 and/or the patient during the procedure.

As shown in FIG. 2, the device 100 can be used with one hand 104 by an operator 106 placing one finger 108 on a guide wheel 110 to operate the guide wheel 110. In at least one example the one finger 108 can be an index finger 302. The operator can grip the system using a middle finger 304 and a thumb 306, while the index finger 302 is free to rotate the guide wheel 110. The grip illustrated in FIG. 2 can be used in the other figures as well, but the views limit the ability to show the grip for clarity purposes. The plastic catheter can be implemented with the system shown in FIGS. 1 and 2 or in some examples, the plastic catheter can be omitted. The distal end of the plastic catheter 120 can be tapered as shown or in other examples can be straight.

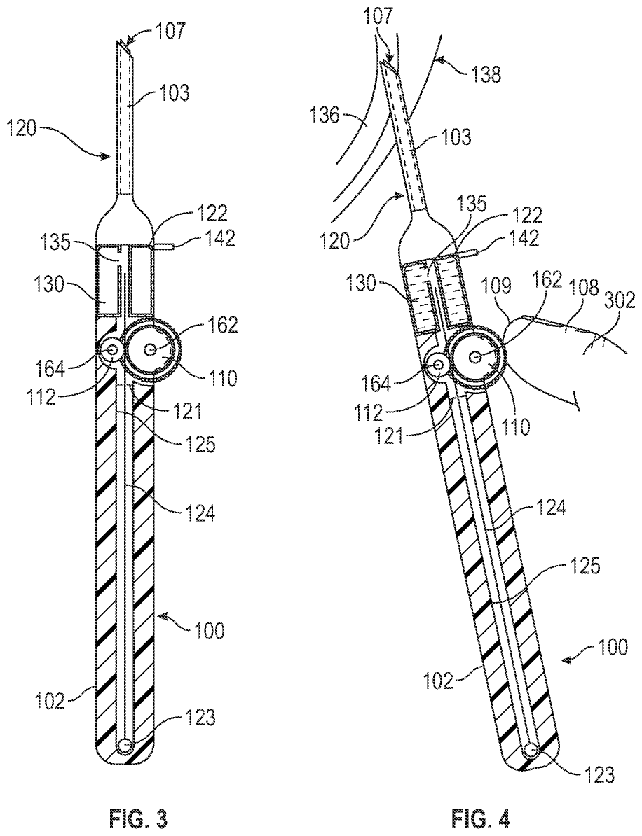

FIG. 3 illustrates the general appearance of the intravenous catheter device 100, primarily in section, with a guidewire 124 built to interact functionally with the guide wheel 110. A plastic catheter 120 is attached to the distal end 122 of the housing 102. In at least one example, the plastic catheter 120 can be detachable from the housing 102. In another example, the plastic catheter 120 can be configured to be releasably retained by the housing 102 thereby preventing separation of the plastic catheter 120 from the housing 102 during normal operation.

A blood flash chamber 130 can be located within the housing 102. The size of the blood flash chamber 130 is for illustrative purposes only. The blood flash chamber 130 can be located at or proximal to the distal end 122 of the housing 102. The blood flash chamber 130 can be much smaller relative to the housing 102 than the one shown. The blood flash chamber 130 can be sufficiently clear and/or translucent to allow the operator 106 to see blood filling the blood flash chamber 130. In at least one example, the blood flash chamber 130 can be integrally formed with the housing 102. In another example, the housing 102 can be formed around the blood flash chamber 130. Blood from a patient flows from the distal end 107 through the needle 103 until it reaches the aperture 135 of the needle 103. An aperture 135 is formed in the blood flash chamber 130. In at least one example, the aperture 135 can be a hole that is substantially round. In other examples, the aperture 135 can take on other shapes such as a square or rectangular shape. The shape of the aperture 135 can be made such that blood freely flows into the blood flash chamber 130.

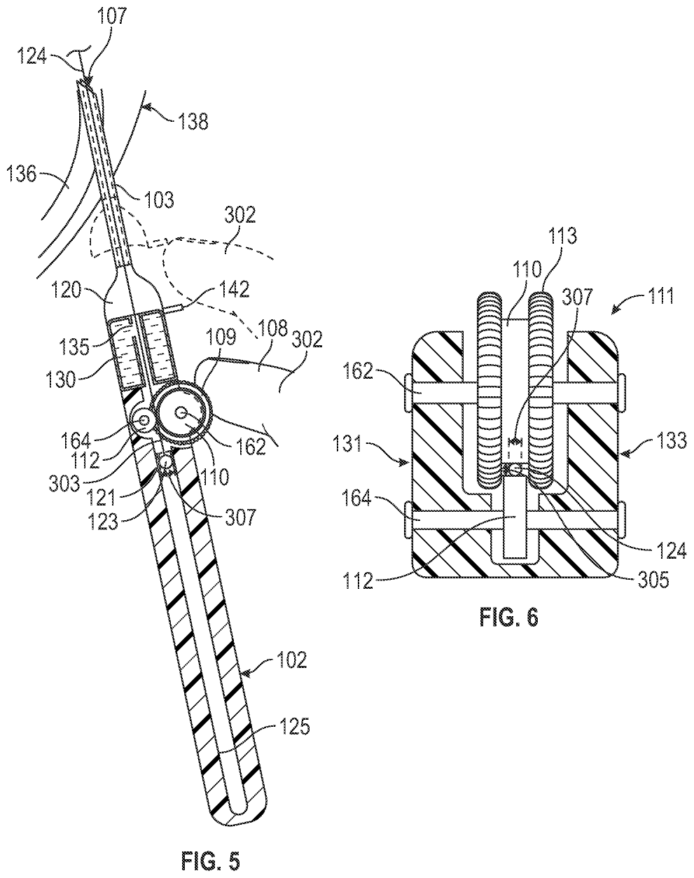

FIG. 4 illustrates inserting the distal end 107 into a vein 136 of a patient. Once the needle 103 has entered the vein, blood flows into the blood flash chamber 130. FIG. 4 further illustrates, once the blood flash is obtained, rotating the guide wheel 110 with the pad 109 of a finger 108. The rotation of the guide wheel 110 causes the guidewire 124 to go from a retracted position to a fully extended configuration as further described in FIG. 5. The needle 103 can fill the blood flash chamber 130 through aperture 135 once the vein 136 is entered by the distal end 107 of the needle 103 since there is no guidewire obstructing blood flow into the chamber 130. Note that the chamber 130 is enlarged in this view relative to the housing 102, but the size of the chamber 130 can vary, while the blood flash function can be representative regardless of the scale to the housing as a flash of blood. A plastic catheter 120 extends over the needle 103 and in at least one example is releasably detachable. Blood return occurs at the aperture 135. This is also a signal to operate the guidewire 124. This can also be a signal to turn the guide wheel 110 and advance the guidewire 124.

FIG. 5 illustrates a configuration in which the guidewire 124 is fully extended in the patient 138. The guidewire 124 exits the distal end 107 of the needle 103, such that a portion of the guidewire 124 is located above the aperture 135. The guidewire is operable to go further into the needle 103 and ultimately into a vein 136. An inner track 125 directs the guidewire 124 into guide wheel 110 and support wheel 112. The guidewire 124 exits from contacting with the guide wheel 110 and support wheel 112 and goes through the needle 103. The ball element 123 disposed on the trailing end 303 of the guidewire 124. Additionally, the ball element 123 centers the guidewire 124 in the track 125 and stops the guidewire 124 from proceeding beyond a ball stop 121 at the end of the track 125. As illustrated, the guidewire 124 is fully advanced into the patient. The advancement of the guidewire 124 is caused by a pad 109 of a finger 108 rotating the guide wheel 110 until the guide wheel stops rotating due to contact of the ball element 123 with ball stop 121. In one example, the ball element 123 can be disposed on a trailing end 303 of the guidewire 124. A distance 305 (shown in FIG. 6) between the guide wheel 110 and support wheel 112 can substantially be the diameter of the guidewire 124. The diameter 307 of the ball element 123 can be substantially larger than the distance 305. In at least one example, substantially larger refers to the ability of the ball element 123 to stop the advancement of the guidewire 124 past the ball stop 121. In at least one example, the diameter 307 of the ball element 123 is at least twice the distance 305.

The operator can use the same finger 108 (for example, an index finger 302) that turned the guide wheel 110 to push on the push off tab 142 to advance the plastic catheter 120 over the guidewire 124 and into the vein 136. As illustrated in FIG. 5, an operator can use a finger 108, for example an index finger 302, to advance the plastic catheter 120 with the help of a push off tab 142. The operator waits until the plastic catheter 120 is within vein 136 to advance the plastic catheter 120 from the retracted position shown in solid lines to the advanced position shown in dashed lines. The guidewire 124 assists in making sure that the plastic catheter 120 stays within the vein of the patient during further insertion by serving as a centering mechanism for the plastic catheter 120. The needle 103 allows for initial penetration of the skin and vein of the patient. When blood enters the blood flash chamber 130, the flash provides for further indication that vein of the patient has been penetrated by the needle 103. The plastic catheter 120 can remain in place to provide for an IV port. The IV port on the end of the plastic catheter 120 allows for insertion of a variety of different IV injections, including but not limited to shots and/or drip bag installations. Once the need for the IV port is no longer, the plastic catheter 120 can be removed.

FIG. 6 illustrates grip locations 131, 133 are on each side of the guide wheel apparatus 111 that includes the guide wheel 110. In at least one example, the grip locations 131, 133 can include a textured surface. The textured surface can include a plurality of ridges and valleys. In another example, the texture surface can be knurled. The textured surface can provide for an enhanced grip on the guide wheel 110 by the operator. The operator will be wearing a glove or have hands that are slippery. The guide wheel apparatus 111 allows the guidewire 124 to pass between the guide wheel 110 and the support wheel 112, which are each mounted to rotate on axles 162 and 164 respectively. While the axles 162 and 164 are shown where they have a portion that is on the outside of the guide wheel apparatus 111, the axles 162 can be molded with guide wheel apparatus 111. As illustrated the guide wheel 110 is operable to rotate about a first axle 162. The support wheel 112 is operable to rotate about a second axle 164. The first axle 162 and the second axle 164 can be arranged to be vertically aligned. Additionally, the first axle 162 and the second axle 164 can be arranged that a line that bisects the center of the first axle 162 and the second axle 164 is perpendicular to the needle 103. Furthermore, in other examples, the exterior portions can be made flush or recessed relative to the guide wheel apparatus 111 so that snags are less likely to happen. The guide wheel 110 and the support wheel 112 and are disposed to frictionally engage the guidewire 124 to move the guidewire 124 forwardly or backwardly in response to movement of the guide wheel 110 by the finger 108 of the operator 106. The part of the guide wheel 110 that has contact with the guidewire 124 can be covered with a plastic or rubber type of material (for example, thermoplastic polyurethane but not limited to this material) that allows the guidewire 124 to be gripped by the guide wheel 110 and prevent slippage of the guidewire 124. In at least one example, the support wheel 112 can have a similar covering.

The guidewire 124 is contained within the device 100. As illustrated the guidewire 124 is located within the housing 102 of the device 100. The guidewire 124 does not extend beyond a portion of the blood flash chamber 130 in the retracted or shipping configuration. Once the device 100 is ready for use, the guidewire 124 can be advanced as described herein to extend beyond the device 100. In the retracted configuration, the guidewire 124 is positioned such that blood can flow through needle 103 into the aperture 135 formed in the blood flash chamber 130. The guidewire 124 can be made of a material such as nitinol or other similar alloy metals that can allow for frictional contact with the guide wheel 110 and support wheel 112 and then be able to be guided through the needle 103 and exit the distal end 107 of the needle 103 into the vein 136. The guidewire 124 can advance past the aperture 135 towards the distal end 107 of the needle 103.

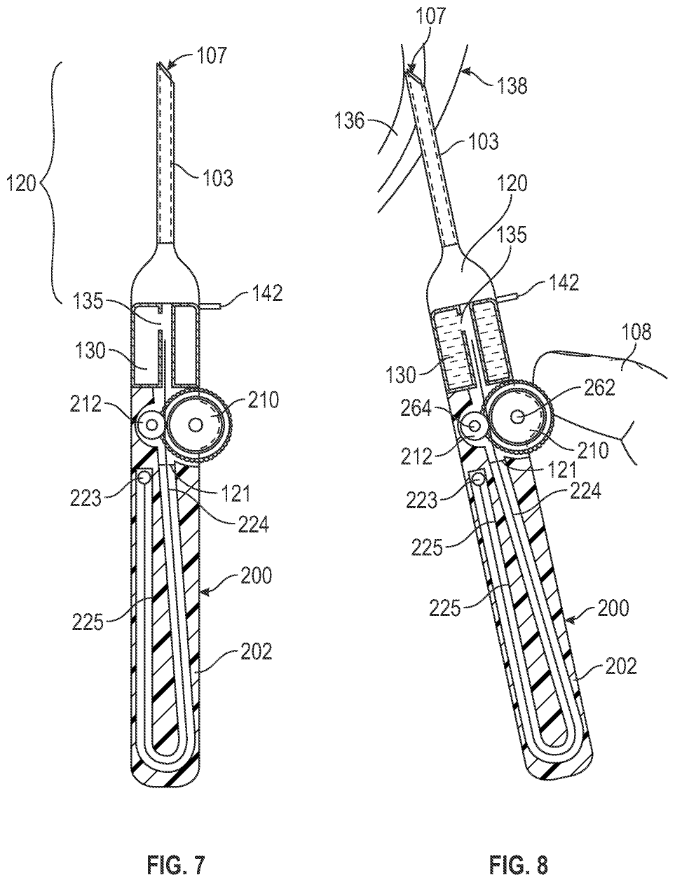

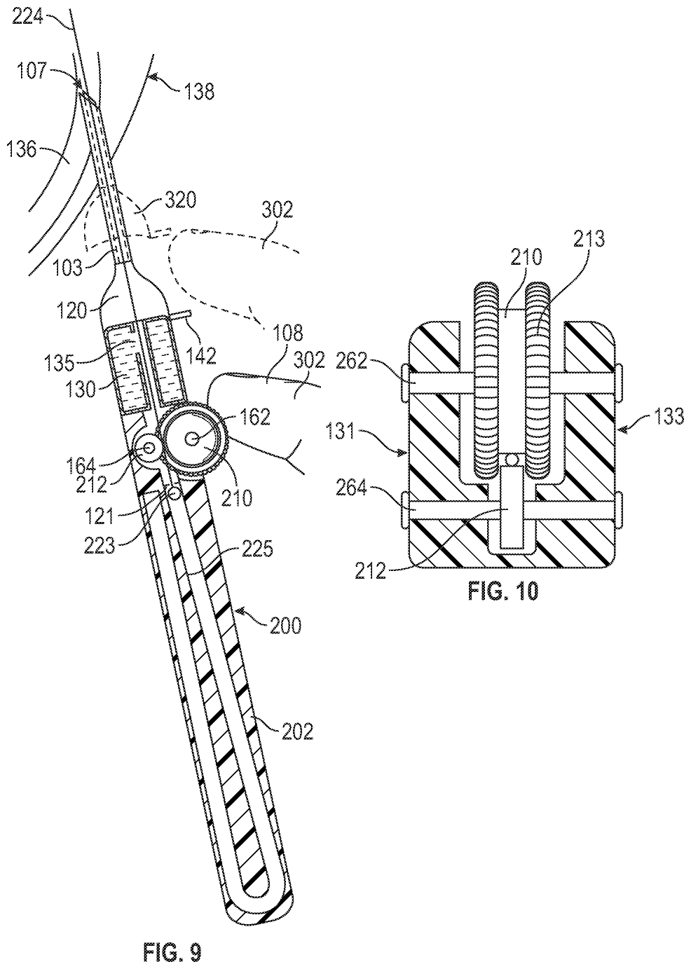

FIGS. 7 to 10 illustrate an alternative example of the system or device 200 where the guidewire 224 is provided a longer length of travel in a shorter length of housing 202 in the same general outer configuration but shorter in length. Thus, the track 225 can be made longer than the length of the housing 202. The track 225 includes a curve that allows for a portion of the housing 202 to have a track 225 that is more than double the length of a straight track 225. The track 225 can include a portion that is substantially parallel to the exterior of the housing 202. Additionally, the track 225 can include a portion that is sloped relative to the exterior of the housing 202. The guide wheel 210 and support wheel 212 can be the same or very similar configuration as the prior example described above, but an extended track 225 is included that adds a distance of travel for the guidewire within the housing 202 and curves within the housing 202 as shown. A ball element 223 is disposed at the end of the guidewire 224 within the track 225 to center the guidewire 224 in the track 225. The ball element 223 is designed such that the ball element is prevented from further advancement by ball stop 121. The ball stop 121 can be formed as a wall with an opening formed therein. In other examples, the track 225 can be molded, printed, and or constructed such that the wall is formed as part of the creation of the housing 202 including the track 225. The guidewire 224 moves between the guide wheel 210 and the support wheel 212 which are each mounted to rotate on axles 262 and 264 respectively and disposed to frictionally engage the guidewire 224 to move the guidewire 224 forwardly or backwardly in response to movement of the guide wheel 210 by the finger 108 of the operator 106. In all other respects, this example would function in a similar manner as that of FIGS. 2 through 6. The guidewire 224 movement would occur after the blood flash appears in the chamber 130 after engagement of the vein 136 (FIG. 8) and subsequent blood flow into the chamber 130 (FIG. 9). The blood flash indicates to the operator that the vein has been penetrated properly. The operator then rotates the guide wheel 210, which engages with the guidewire and advances the guidewire through the needle 103 and out the distal end 107 of the needle 103. Once the guidewire 224 is advanced the full length, the operator then slides the plastic catheter 120 into the vein. The plastic catheter 120 is guided by the guidewire 224. Once the plastic catheter 120 is installed, the device 200 can be removed leaving the plastic catheter 120 in place in the patient 138. In the illustrated example of FIG. 9 relative to FIG. 5, the track length is increased. In other examples, the track length can remain the same, but the housing of the device can be reduced accordingly.

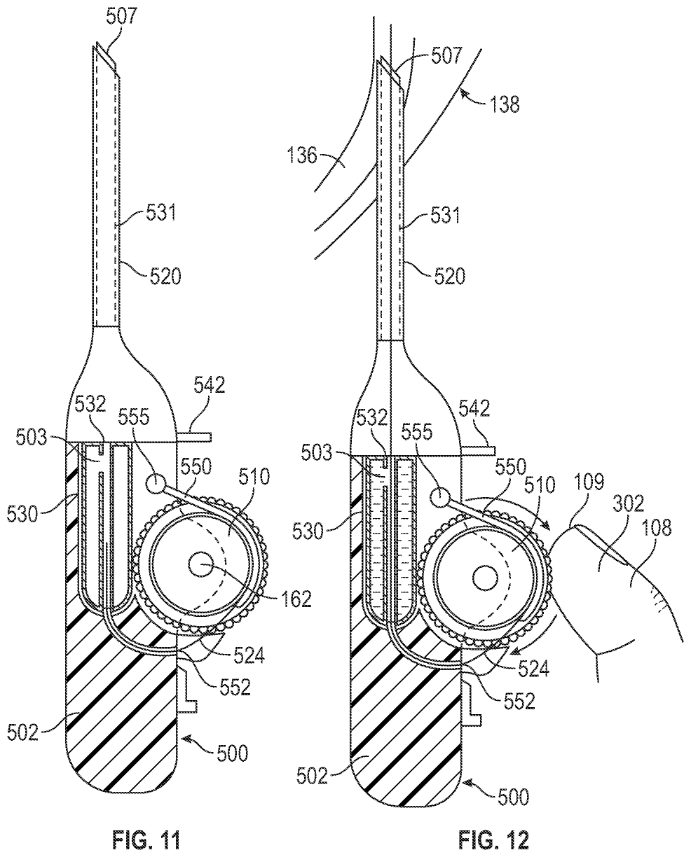

FIGS. 11 through 14 disclose a further example of the disclosure having a shorter length housing 502 and a guide wheel 510 in which the guidewire 524 wraps around the guide wheel 510 as it is manipulated by the finger 108 of an operator 106. FIG. 11 illustrates the general appearance of the intravenous catheter guidewire system or device 500 with a guidewire 524 built into the guide wheel 510. The guidewire 524 wraps around the guide wheel 510 multiple times with the end of the guidewire positioned so as to not pass the aperture 503 in a retracted configuration. The guidewire 524 is positioned in the retracted or original configuration such that the guidewire 524 wraps several times around the guide wheel 510. The guidewire 524 is coupled to the guide wheel 510 by a tensioner 550. The guidewire 524 continues through a passageway 552 formed in the housing 502. The guidewire 524 continues through at least a portion of the needle 531 and past the distal end 507 of the needle 531.

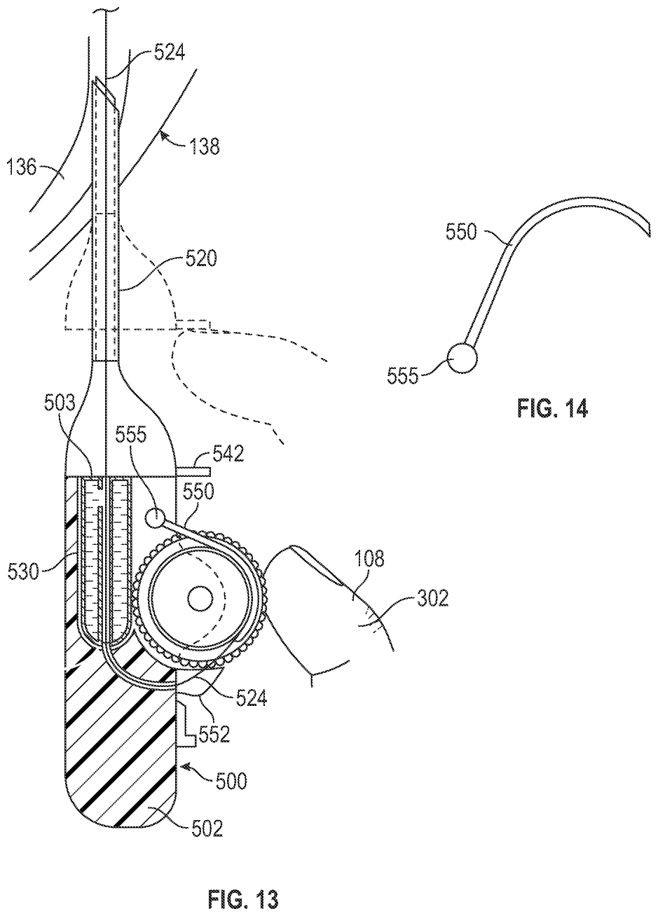

When the operator inserts the device 500 into the patient 138, the distal end 507 of the needle 531 is configured to penetrate the patient and the vein of the patient. As the guidewire 524 is in the retracted state, blood can flow through the needle 531 into the blood flash chamber 530. The blood flash chamber 530 is shown as a clear plastic portion of the housing 502 that is visible to an operator 106 and that should fill with an obvious blood flash since there is no guidewire obstructing blood flow into the chamber 530 via an aperture 503. The guidewire 524 is originally disposed just inside the needle 534 above the aperture 503 located in the needle 534 (FIG. 11). In at least one example the passageway 532 The guidewire 524 moves from the guide wheel 510 into a passageway 552 in the housing 502 into the needle 534. Blood return occurs at the aperture 503 and should cause a blood flash once the distal end 507 of needle 531 is inside the vein 136 of a patient 138 (the guidewire 524 blocks return through the end of the needle 534). A push off tab 542 is connected to the plastic catheter 520 and assists with pushing the catheter 520 into the vein 136 and disconnecting with the housing 502 with one finger 108 after the guidewire 524 has been advanced as shown in FIG. 13.

A spring loaded tensioner 550 is fixed to the housing at one end via a securement element 555 and wrapped around the guidewire 524 on the guide wheel 510 to control the radial position of the guidewire 524 on the guide wheel 510 as it is reeled in or out during movement of the guide wheel 510 and relative movement of the guidewire 524. The guidewire 524 is wrapped around the guide wheel 510 more than one time, generally, and is secured to the guide wheel 510 at the end opposite to the end entering the vein 136. This construction permits more linear travel of the guidewire 524 in a shorter length of housing 502 to obtain the same results as described above. The tensioner 550 is shown separately in FIG. 14 and would conventionally be made from a spring steel material, preferable stainless steel, with a securement element 555 either welded to the steel if a metal stop or molded to the steel if a plastic element. The guide wheel 510 may be larger in diameter than the guide wheels of the other examples to accommodate more travel for fewer turns of the guide wheel 510 as the guidewire 524 wraps around the guide wheel 510.

In this case the guidewire 524 will be covered by a plastic material (but not limited to plastic material) to help keep it sterile. This covering will protect the guidewire 524 from the operator's gloved finger 108.

It should also be noted that with slight modifications in needle length and types of plastic catheters, the disclosure can be used with central and arterial lines in the same manner as described above.

The instant disclosure has been described in an illustrative manner, and it is to be understood that the terminology which has been used is intended to be in the nature of words of description rather than of limitation. Obviously, many modifications and variations of the instant disclosure are possible in light of the above teachings. It is, therefore, to be understood that within the scope of the appended claims, the instant disclosure may be practiced otherwise than as specifically described.

What is claimed is:

1. An intravenous catheter device comprising:
a housing;
a guidewire completely disposed within the housing;
a guide wheel disposed in the housing and operable to rotate about a first axle, the guide wheel comprising an inner surface configured to engage the guidewire and move the guidewire;
a support wheel operable to engage the guidewire opposite the guide wheel, wherein the support wheel is operable to rotate about a second axle.

2. The intravenous catheter device as recited in claim 1, wherein the housing defines a track for receiving the guidewire.

3. The intravenous catheter device as recited in claim 2, further comprising a ball element disposed on a trailing end of the guidewire that centers the guidewire in the track and contacts a ball stop formed within the track and proximal to the guide wheel.

4. The intravenous catheter device as recited in claim 2, wherein the track includes a portion that curves such that track is longer than a length of the housing.

5. The intravenous catheter device as recited in claim 4, wherein the track includes a portion that is substantially parallel to an exterior of the housing.

6. The intravenous catheter device as recited in claim 4, wherein the track includes a portion that is sloped relative to an exterior of the housing.

7. The intravenous catheter device as recited in claim 1, further comprising a needle extending from a distal end of the housing and operable to receive the guidewire therethrough.

8. The intravenous catheter device as recited in claim 7, further comprising a passageway extending from a distal end of the needle to a second end located within the housing.

9. The intravenous catheter device as recited in claim 8, further comprising a chamber located within the housing.

10. The intravenous catheter device as recited in claim 9, wherein the chamber forms an aperture to the passageway.

11. The intravenous catheter device as recited in claim 10, wherein the aperture is located proximal to the distal end of the housing.

12. The intravenous catheter device as recited in claim 9, wherein the chamber is visible through the housing, such that when blood enters the chamber, a blood flash can be visible to an operator.

13. The intravenous catheter device as recited in claim 7, further comprising a plastic catheter operable to fitted over the needle.

14. The intravenous catheter device as recited in claim 13, wherein the plastic catheter comprises a tab that extends therefrom.

15. The intravenous catheter device as recited in claim 13, wherein the plastic catheter is operable to slide away from the distal end of the housing while the needle remains fixed.

16. The intravenous catheter device as recited in claim 1, wherein the guide wheel further comprises an outer surface radially spaced from the inner surface and having a textured surface.

17. The intravenous catheter device as recited in claim 16, wherein the textured surface includes a plurality of ridges and valleys.

18. The intravenous catheter device as recited in claim 17, further comprising a ball element disposed on a trailing end of the guidewire; a track formed in the housing; and a ball stop formed at distal end of the track, such that the ball element centers the guidewire in the track and the ball stop prevents the guidewire from further advancing once the ball element contacts the ball stop.
